(19) United States
(12) Patent Application Publication
(10) Pub. No.: US 2006/0089676 A1
(43) Pub. Date: Apr. 27, 2006

(54) CARDIAC PACEMAKER WITH LOSS OF ATRIAL CAPTURE MODE SWITCHING AND METHOD

(76) Inventors: William B ROTTENBERG, Durango, CO (US); Scott C MATHIS, Durango, CO (US)

Correspondence Address: JOHN RICHARD MERKLING, 11171 WEST EXPOSITION DRIVE, LAKEWOOD, CO, 80226-3867, US

(21) Appl. No.: 11/163,482

(22) Filed: Oct. 20, 2005

Related U.S. Application Data
(60) Provisional application No. 60/620,484, filed on Oct. 21, 2004.

(51) Int. Cl. A61N 1/362; A61N 1/36

(52) U.S. Cl. 607/14

(57) ABSTRACT

An apparatus and method for treatment of pacemaker mediated tachycardia. A pacemaker detects serially recurring pacemaker mediated tachycardia and alters the mode of stimulation to a non-atrial pacing mode. The pacemaker also alters the mode of stimulation to a non-atrial pacing mode whenever atrial loss of capture is indicated, for example by failure to detect any non-atrial loss-of-capture indicators, or whenever pacemaker mediated tachycardia occurs immediately following atrial stimulation.

This application claims the benefit of U.S. provisional application 60/620,484, filed Oct. 21, 2004.

### BACKGROUND OF THE INVENTION

This invention pertains to a method and apparatus for applying cardiac stimulation, and more particularly, to a method and apparatus for addressing pacemaker mediated tachycardia (PMT) by mode switching in response to loss of atrial capture.

The heart is a mechanical pump that is stimulated by electrical impulses. The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV), which then pumps it throughout the body. Four heart valves keep the blood flowing in the proper directions.

The electrical signal that drives this mechanical contraction starts in the sino-atrial node, a collection of specialized heart cells in the right atrium that automatically depolarize (change their voltage potential). This depolarization wave front passes across all the cells of both atria and results in atrial contraction. When the advancing wave front reaches the A-V node, it is delayed so that the contracting atria have time to fill the ventricles. The depolarizing wave front then passes over the ventricles, causing them to contract and pump blood to the lungs and body. This electrical activity occurs approximately 72 times a minute in a normal individual and is called normal sinus rhythm.

The corresponding electrical signals identifying these events are usually referred to as the P, QRS (or R) and T waves or beats. More particularly, an atrial contraction is represented on an ECG by a P wave, a ventricular contraction is represented by an R wave and a ventricular repolarization is represented by a T wave. The atrium also repolarizes but this event (the U wave) is masked by activity in the ventricle and consequently it is not observable on an ECG. When the physiologic mechanisms that produce normal sinus rhythm fail, a cardiac pacemaker may be implanted to restore cardiac rhythm. Pacemaker mediated tachycardia (PMT) is a well-known phenomenon that can occur with dual chamber cardiac pacemakers.

PMT is an endless loop phenomenon whereby if an implanted cardiac pacemaker delivers an unsynchronized ventricular pacing pulse, retrograde cardiac conduction can cause atrial activation (retrograde P wave). This retrograde P wave can be detected by the pacemaker and trigger another ventricular pacing pulse in response to the sensed atrial activity. This triggered behavior is the normal and desired behavior of a modern dual chamber pacemaker, but if the atrial activity is due to retrograde conduction, a non-physiologic loop behavior can be sustained by the pacemaker. The triggered ventricular pulse results in another retrograde P wave and the cycle continues. Normally, retrograde conduction does not occur because the AV conduction system is refractory for a short period after a synchronized ventricular event due to the antegrade conduction. Unsynchronized ventricular events can initiate retrograde conduction because the AV conduction system was not activated prior to the event and, not being refractory, can be excited by the ventricular event. Retrograde conduction is present in the majority of pacemaker patients.

Because PMT is a highly undesired clinical behavior, most modern cardiac pacemakers have mechanisms to detect and terminate the behavior. In most cases, when the device recognizes a regular and high atrial rate that is tracked by ventricular pacing, it simply withholds one ventricular, triggered pacing pulse. This breaks the cycle and the following cardiac event should be another synchronized cycle.

There are numerous events that are known to initiate PMT, and many of them have some signature that the pacemaker can recognize and use to prevent the resulting PMT. For example a premature ventricular contraction (PVC) can initiate retrograde P waves and start the same loop, but the pacemaker can recognize a PVC and not track the next atrial event if it is too soon. Techniques like this are also well understood by those in the field.

Loss of atrial capture is also well known to cause PMT because the following ventricular event will most likely be an unsynchronized ventricular event due to the lack of prior atrial pacing. Loss of atrial capture, however, is difficult for the pacemaker to detect. This is a very desirable feature and much time has been invested trying to develop a system that can detect loss of atrial capture.

Although there are many initiators of PMT, loss of atrial capture is a common initiator. Since PMT is an undesired clinical behavior, it would be advantageous if a cardiac pacemaker could recognize loss of atrial capture and alter its behavior in some manner to prevent recurring PMT. Changing the pacing modality to one not subject to PMT in the presence of loss of atrial capture would be an effective mechanism to handle this clinical condition.

U.S. Pat. Nos. 6,618,622; 6,285,908; 6,259,950; and 6,243,606, all held by St. Jude Medical, each discuss various indirect atrial capture determination methods and includes PVARP extensions or simultaneous atrial/ventricular pulses following loss of atrial capture to prevent PMT. The St. Jude patents discuss temporary PMT prevention following loss of atrial capture during an atrial pacing threshold test.

U.S. Pat. No. 5,674,255, by Guidant, describes an automatic PVARP adjustment to prevent repetitive PMT episodes. This patent differs from the St. Jude ones in that it includes methods to permanently prevent PMT. The Guidant patent uses measured retrograde conduction times to adjust the programmed PVARP value. Normally, the PVARP is intended to be just long enough to prevent atrial sensing of any retrograde P waves. Making it too long limits atrial tracking capabilities. Systems desirably use the shortest possible PVARP and use extensions to the PVARP in the presence of known PMT initiators to block atrial sensing of retrograde P waves. While this system would achieve the same end result of prevention of PMT, it would also severely limit the device's atrial tracking capability by extending the PVARP for too long.

Therefore, it would be desirable for an implantable cardiac pacemaker that can recognize uncorrectable loss of atrial capture to change its pacing modality to one that is not subject to PMT.

### SUMMARY OF THE INVENTION

In view of the above disadvantages of the prior art, it is an objective of the present invention to provide an implantable cardiac stimulation system, such as a pacemaker, in which various responses could be implemented once a cardiac pacemaker suspects loss of atrial capture that cannot be corrected with increased atrial output energy. The responses can be temporary, such as would result from an occasional loss of atrial capture, or permanent if a consistent loss of atrial capture is detected.

Loss of atrial capture can be determined in several ways. One method would follow methods similar to detecting loss of ventricular capture: measuring some electrical or mechanical signal that results from a cardiac contraction following a pacing pulse. Other published methods to suggest atrial capture include detection of the resulting AV conduction or the detection of a synchronized intrinsic ventricular contraction.

In response to detection of the loss of atrial capture, the cardiac pacemaker would initiate a mode change to ventricular stimulation modes, either VDD or VVI mode, with or without rate response. Either mode eliminates atrial pacing, thus removing the chance for loss of atrial capture to initiate a PMT and eliminating the wasted energy of the ineffective atrial pacing pulse.

### BRIEF DESCRIPTION OF THE DRAWINGS

FIG. 1 shows a diagrammatic front view of a patient with a cardiac stimulation system, including a programmer used to program the cardiac stimulator.

FIG. 2 shows a block diagram of the cardiac stimulator of FIG. 1.

FIG. 3 is a flow chart for declaring loss of capture.

FIG. 4 is a flow chart for changing mode in the presence of atrial capture loss.

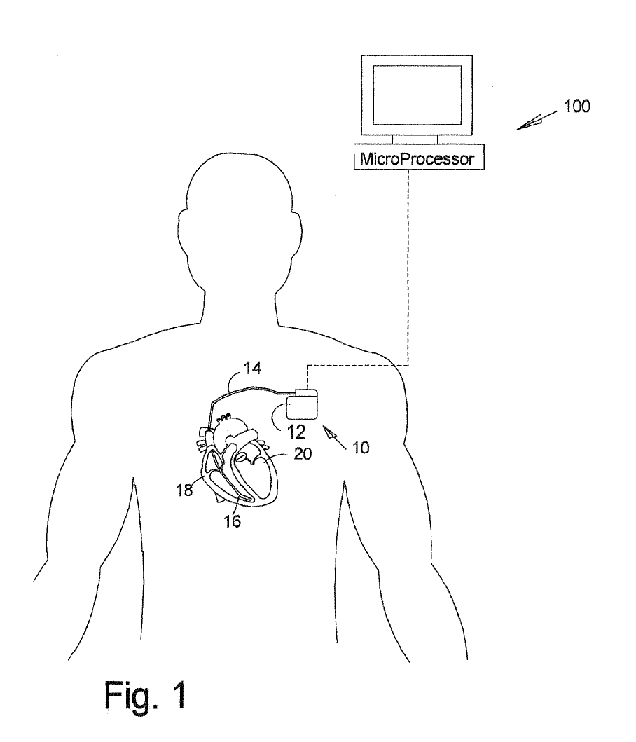

### DETAILED DESCRIPTION OF THE INVENTION

The subject invention pertains to an implantable cardiac stimulation system 10 including a cardiac stimulator 12 with various electronic circuits, and a multi-electrode lead 14 attached to the stimulator 12, as shown. The lead 14 has a distal end 16 disposed, for example, in one of the cardiac chambers such as the right ventricle 18 of heart 20. The system 10 is adapted to deliver therapy in the form of electrical pulses. The cardiac stimulator 12 contains electronic components common to current cardiac stimulators such as a battery, microprocessor control circuit, ROM, RAM, an oscillator, reed switch and antenna for communication, output circuits, and sense circuits. These components are well known to those of skill in the art. In addition the cardiac stimulator 12 has sensing and stimulating circuits for each at least the right atrium and the right ventricle.

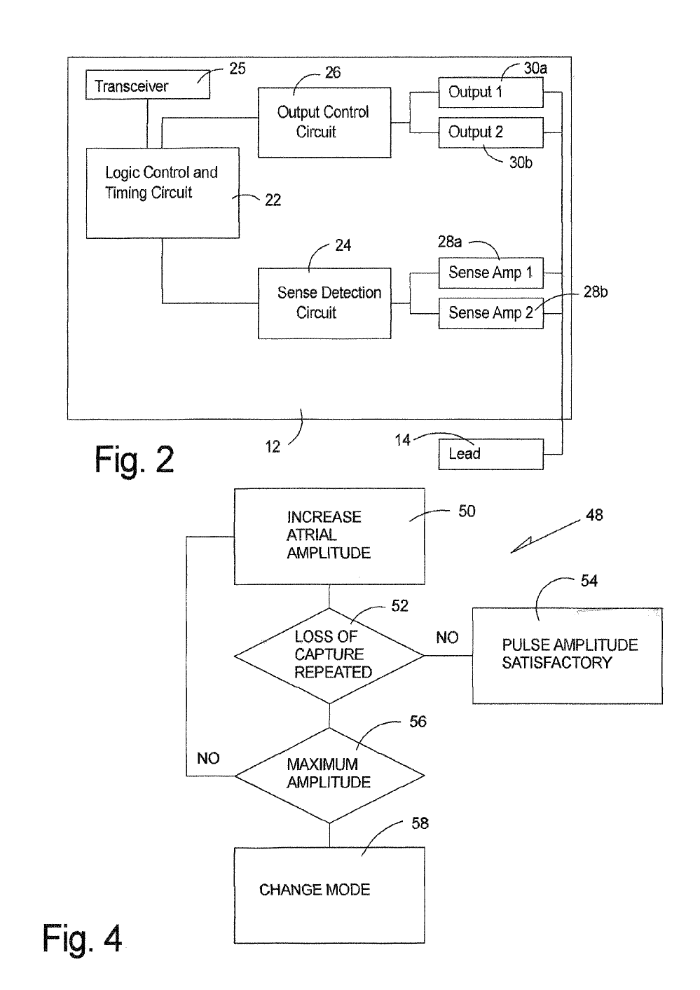

FIG. 2 illustrates important elements of the cardiac stimulator 12 in block diagram. The cardiac stimulator 12 comprises a logic control and timing circuit 22, which may include a microprocessor and memory, but which could also be implemented in a specialized circuit. The logic control and timing circuit 22 receives input from a sense detection circuit 24 and issues control instructions to an output control circuit 26. To accommodate the electrodes used in the apparatus, at least two sense amplifiers 28a, 28b are provided, each in electrical communication with an electrode through the lead 14 and with the sense detection circuit 24. Similarly, the output control circuit 26 is electrically connected to at least two output circuits 30a, 30b. The output circuits 30a, 30b produce stimulating pulses at electrodes in the heart through the lead 14. The logic control and timing circuit 22 may operate in accordance with a program stored into memory. The programming in memory is received through a transceiver 25 (for instance from programmer 100). The sensing detection circuit 24 senses intrinsic activity and other signals within the heart 20 and provides corresponding indication signals to the microprocessor. The Logic control and timing circuit 22 then issues appropriate commands to the output control circuit 26. The output control circuit 26 generates appropriate stimulation pulses. These pulses are steered to the electrodes in the atrium or the ventricle.

This invention describes cardiac pacemaker responses to loss of atrial capture. Since consistent loss of atrial capture can result in continuous PMT, as well as wasted energy, the described responses prevent either occurrence.

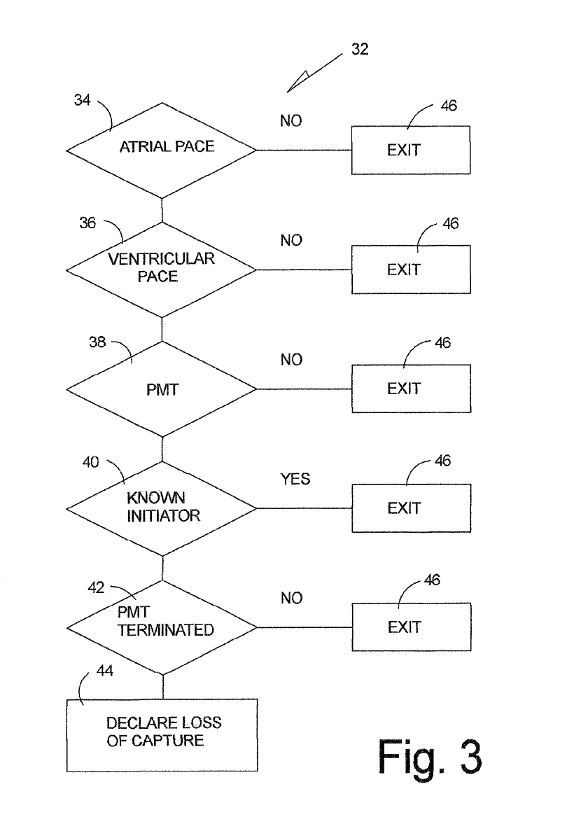

A preferred method 32 for detecting loss of atrial capture is disclosed (see FIG. 3) but other methods to determine loss of atrial capture may be used. For example, one method to detect loss of atrial capture is the consistent initiation of PMT 38 following atrial pacing 34 and no ventricular contraction 36. Current technologies reliably identify PMT with very good certainty. In particular, the system used by St. Jude Medical identifies eight consecutive V-A intervals that are within a certain variance to trigger one dithered A-V interval. In the dithered interval, the A-V interval is changed and the resulting V-A interval is measured. If the V-A interval remains constant, it is deemed to be linked to the ventricular event. If the interval changes by the same amount as the dither, it is actually an atrial rate. There are very few physiologic or extraneous events that could mimic true V-A conduction. If, however, more specificity is desired, additional dither intervals could be added.

When determining repetitive PMT initiation, as would be consistent with loss of atrial capture, one would expect another PMT to initiate within a few cardiac cycles of termination of the prior PMT. A count that determines how fast the PMT re-initiates can be used to improve selectivity. Another way to implement this repetitive measure could be to specify X repetitions within Y cycles.

If false triggers are suspected, a monitor mode can be available where the device collects detailed data regarding intended responses, but does not actually implement them. This can be useful to troubleshoot and fine-tune the behavior of the algorithm.

An important property of the algorithm's specificity is the recognition 40 of other known initiators of PMT and not considering them loss of atrial capture. Other known initiators include PVCs, magnet mode termination and noise reversion termination. Both magnet mode termination and noise reversion termination are known to the device, so the algorithm can be disabled for that cycle. PVCs do not follow atrial pacing, so the algorithm can also be disabled easily. High atrial rates can cause the device to track and possibly declare a PMT present. However, atrial pacing is required to be present before loss of atrial capture can occur and initiate a PMT. Therefore, high atrial rates that do not initiate immediately and suddenly following an atrial pulse can be ruled out. Additionally, the rate stability and dithering characteristics of the PMT algorithm can rule out atrial rates. A final test 42 for termination of PMT is performed before loss of atrial capture is declared 44. If any of the conditions (atrial pacing, ventricular contraction, PMT existing, no known alternative initiator or termination of PMT), the algorithm exits 46 and there is no loss of capture.

Responses 48 (see FIG. 4) to loss of atrial capture can include increasing the atrial stimulation amplitude 50 in an attempt to regain capture. If capture loss is not repeated 52, the new pulse amplitude is deemed satisfactory 54. Otherwise, the applitude is increased 50 until a maximum amplitude 56 is reached. If re-capture is unsuccessful, the pacemaker 12 changes mode 58 to a non-atrial pacing mode (VDD or VVI mode). The termination of atrial pacing prevents the possibility of a PMT initiating following loss of atrial capture, as well as eliminating the wasted energy of atrial pacing that is not producing an atrial event.

The mode change can be temporary if the loss of capture appears to be transient or intermittent. This could be the case if the loss of capture were due to an electrolytic change that results in a temporarily higher pacing threshold, or as a result of some atrial pacing threshold search algorithm that will, by its nature, lose atrial capture. In these cases, the mode switch can be for one cycle. If, on the other hand, consistent loss of atrial capture is detected, a permanent mode change can be initiated. Occasional searches for atrial capture can be made by temporary mode changes back to the original mode. In either case, an additional diagnostic can be added to the system that would indicate the mode change and the suspicion of an atrial capture problem.

One additional advantage to disabling atrial pacing when it is ineffective is the preservation of battery life by eliminating the useless atrial pacing pulses. In fact, a threshold tracking system that might deliver a high-energy pulse to try to regain capture can waste significant energy.

A novel method of loss of atrial capture detection is described in this invention, as well. Many modern pacemakers can recognize and terminate a PMT. In addition, loss of atrial capture can result in a PMT. If a PMT is detected immediately (that is, within five cardiac pace cycles or less) following an atrial pulse, it is reasonable to assume that the atrial pacing pulse did not capture. Further, if the pacemaker terminates the PMT and it immediately reinitiates, it is more likely that loss of atrial capture is consistently initiating PMT. Thus loss of capture may be declared after a selected number of cycles detection of PMT, followed by PMT termination, followed by re-detection of PMT within a short period of time or immediately after termination. This can be implemented in connection with increasing amplitude of atrial pulse 56, as shown in FIG. 4. While most current ventricular capture detection methods, and probably most new atrial capture detection methods, require specific circuitry to make the detection, this algorithm can be implemented with existing circuitry. This technique only works if the patient has retrograde conduction, which is present in the majority of patients.

Numerous other modifications may be made to this invention without departing from its scope as defined in the attached claims.

What is claimed is:

1. A cardiac pacemaker comprising
an atrial sensor,
a ventricular sensor,
an atrial stimulator,
a ventricular stimulator,
a controller electrically coupled to the sensors and stimulators said controller comprising means for detecting serially recurring pacemaker mediated tachycardia and for altering the mode of stimulation for said pacemaker to a non-atrial pacing mode whenever a selected number of pacemaker mediated tachycardias recur within a selected period of time.

2. The cardiac pacemaker according to claim 1, wherein a pacemaker mediated tachycardia recurs immediately after termination of a preceding pacemaker mediated tachycardia.

3. The cardiac pacemaker according to claim 1 further comprising means for testing for non-atrial loss-of-capture initiators of pacemaker mediated tachycardia, and for altering the mode of stimulation for said pacemaker to a non-atrial pacing mode whenever atrial loss of capture is indicated by failure to detect any non-atrial loss-of-capture indicators.

4. The cardiac pacemaker according to claim 1 further comprising means for determining that a pacemaker mediated tachycardia has occurred immediately after an atrial stimulation has been delivered and for altering the mode of stimulation for said pacemaker to a non-atrial pacing mode whenever said pacemaker mediated tachycardia occurs immediately following an atrial stimulation.

5. A cardiac pacemaker comprising
an atrial sensor,
a ventricular sensor,
an atrial stimulator,
a ventricular stimulator,
a controller electrically coupled to the sensors and stimulators said controller comprising means for detecting atrial loss of capture, and for altering the mode of stimulation for said pacemaker whenever atrial loss of capture is indicated.

6. The cardiac pacemaker according to claim 5 further comprising means for testing for non-atrial loss-of-capture initiators of pacemaker mediated tachycardia, and for altering the mode of stimulation for said pacemaker to a non-atrial pacing mode whenever atrial loss of capture is indicated by failure to detect any non-atrial loss-of-capture indicators.

7. The cardiac pacemaker according to claim 6 further comprising means for detecting pacemaker mediated tachycardia.

8. A cardiac pacemaker comprising
an atrial sensor,
a ventricular sensor,
an atrial stimulator,
a ventricular stimulator,
a controller electrically coupled to the sensors and stimulators said controller comprising means for detecting pacemaker mediated tachycardia, means for determining that a pacemaker mediated tachycardia has occurred immediately after an atrial stimulation has been delivered and for altering the mode of stimulation for said pacemaker to a non-atrial pacing mode whenever said pacemaker mediated tachycardia occurs immediately following an atrial stimulation.
